(19) United States
(12) Patent Application Publication
(10) Pub. No.: US 2018/0333051 A1
(43) Pub. Date: Nov. 22, 2018

(54) FLEXIBLE MEMBER FOR VITAL SIGN MEASUREMENT AND METHOD THEREOF

(71) Applicant: Wadeeah PATRICK-CARY, Philadelphia, PA (US)

(72) Inventor: Wadeeah PATRICK-CARY, Philadelphia, PA (US)

(21) Appl. No.: 15/599,882

(22) Filed: May 19, 2017

(51) Int. Cl. A61B 5/00; A61B 5/04; A61B 5/0205

(52) U.S. Cl. CPC A61B 5/0002; A61B 5/04; A61B 5/0024; A61B 5/6802; A61B 5/0205; A61B 5/0008; A61B 5/0022

(57) ABSTRACT

A vital sign measurement apparatus includes a flexible member having an interior, wherein the flexible member includes a pocket. The pocket includes a closure mechanism for closing the pocket maintaining the pocket in a closed position. The closure mechanism can be open for accessing an interior of the pocket. A wireless transmitting device is located in the pocket and is removable from the pocket upon opening the closure mechanism. Detectors are included for measuring at least one of respiration, temperature, heart rate, and blood pressure. The wireless transmitting device receives signals that include data corresponding to at least one of the measured respiration, the temperature, the heart rate, and the blood pressure. The wireless transmitter then provides a wireless signal to a microprocessor-based computer system that is external to the flexible member based on the measured respiration, temperature, heart rate, and blood pressure.

### FIELD OF THE INVENTION

The present invention relates to vital sign measurement and in particular to an apparatus and method that enables one or more types of vital measurement to occur. Specifically, a flexible member is disclosed, such as cloth, which may be used in combination with a method or apparatus for vital sign measurement.

### BACKGROUND OF THE INVENTION

The medical field provides various forms of care in order to address various health needs. One example is primary care which has several goals, including preventing disease through regular physical exams and diagnosing and treating a wide variety of conditions. Another form of care is specialty care which is provided to a patient when special knowledge in one or more medical areas is required. A further form of care is emergency care which is provided to a patient when a life-threatening illness or injury requires immediate medical attention. Yet another form of care is urgent care which is provided to a patient when an illness or injury requires immediate attention even though it is not urgent. Long-term care is another form of care that may be necessary when a patient has an ongoing medical condition.

Depending upon the type of care, the patient may need to go to various facilities including the office of a primary care physician, the office of a specialty care physician, an urgent care center, a hospital emergency room, a hospital bed facility, etc.

Regardless of the type of care that is provided to a patient, it is often desirable (and sometimes necessary) to obtain a patient's vital signs. Vital signs are often obtained as the first step towards administering any type of medical care to a patient. Examples of vital signs include body temperature, respiration (level and/or rate) heart rate (pulse) and blood pressure. As a patient is receiving care, vital signs may be obtained using a combination of various machines. Blood pressure, for example, can be obtained using a sphygmomanometer or other type of blood pressure detection device. Body temperature may be obtained using an electronic thermometer, by placing its tip under the patient's tongue. Respiration can be measured, for example, using pulse oximetry. Heart rate maybe measured using a device as complex as a heart rate monitor, or as simple as feeling a pulse near the patient's wrist and timing the pulse using the second hand of a watch.

When vital signs are measured as a first step in preventative care or diagnosis, it is possible to measure one vital sign it at a time. Thus, for example, a healthcare practitioner can measure body temperature and then measure blood pressure. Alternatively, it may be possible for a healthcare practitioner to measure several vital signs at once. In a hospital bed, however, or more particularly in an intensive care unit, it may be desirable to measure several vital signs at once, and it may be desirable for the measurement of multiple vital signs to be simultaneous and ongoing. In such a situation, numerous devices may be attached to a patient at one time so that multiple vital signs can be measured simultaneously.

### SUMMARY OF THE INVENTION

A vital sign measurement apparatus includes a flexible member having an interior, wherein the flexible member includes a pocket. The pocket includes a closure mechanism for closing the pocket maintaining the pocket in a closed position. The closure mechanism can be open for accessing an interior of the pocket. A wireless transmitting device is located in the pocket and is removable from the pocket upon opening the closure mechanism. Detectors are included for measuring respiration, temperature, heart rate, and blood pressure. The wireless transmitting device receives signals that include data corresponding to the measured respiration, the temperature, the heart rate, and the blood pressure. The wireless transmitter then provides a wireless signal to a microprocessor-based computer system that is external to the flexible member based on the measured respiration, temperature, heart rate, and blood pressure. The flexible member may be a cloth member, or may be part of a cloth member, such as a blanket or pillow. An exterior surface of the flexible member may permit objects, such as toys, to be affixed thereto.

### BRIEF DESCRIPTION OF THE DRAWINGS

FIG. 1 is a perspective drawing that illustrates a flexible member in accordance with an exemplary embodiment of the present invention.

FIG. 2 is a block diagram that illustrates exemplary detectors for measuring vital signs in accordance with an exemplary embodiment of the present invention.

FIG. 3 is a block diagram that illustrates exemplary optional features that may be included with exemplary flexible members disclosed herein.

FIG. 4 is a block diagram that illustrates communication between an exemplary measurement apparatus and exemplary microprocessor based devices in accordance with one or more exemplary embodiments of the present invention.

FIG. 5 is a block diagram of an exemplary electronic circuit that may be used for measuring body temperature.

FIG. 6 is a block diagram of an exemplary electronic circuit that may be used for measuring blood pressure.

FIG. 7 is a block diagram of an exemplary electronic circuit that may be used for measuring heart rate.

FIG. 8 is a block diagram of an exemplary embodiment of the present invention that illustrates multiple detectors partially separated from, but connected to, the exemplary flexible member.

FIG. 9 is a perspective drawing that shows how optional belt-like devices may be used for sensing vital sign data.

FIG. 10 illustrates how an exemplary flexible member can be used with other objects, such as toys, attached thereto.

### DETAILED DESCRIPTION

When providing a patient with medical care, it is often very desirable to first measure vital signs. As previously explained, there are a plurality of vital signs that are typically measured, including heart rate, blood pressure, respiration, and body temperature. Multiple devices may be used in order to measure these various vital signs. Often, for example, the devices used for such measurement need to be attached to the patient in various ways. For example, blood pressure is often measured by placing a cuff around a patient's arm. Respiration is often measured by placing a pulse oximetry device on the patient's finger. Sometimes, pulse is also measured using the pulse oximetry device. Body temperature may be measured by placing the sensor of electronic thermometer under a patient's tongue. When a patient has several devices attached to them at once, this can be both physically uncomfortable and emotionally concerning. With children in particular, children are very often nervous simply as a result of being in a doctor's office or hospital. When multiple devices are attached to a child simultaneously, a child may find the situation to be not only uncomfortable, but even stressful or disturbing.

It is thus desirable to be able to measure the vital signs of the patient and at the same time to minimize stress and or upsetness that is caused to the patient as a result of measuring their vital signs. With children in particular, it is highly desirable to take appropriate steps to minimize stress and upset mess when measuring vital signs. This objective becomes even more important if the vital signs must be taken over a long period of time, such as during a hospital stay. If devices to measure vital signs need to be attached to a patient over a long period of time, it becomes even more important to try to take steps to reduce the patient stress and upset us. Again, this need becomes even greater when the patient is a child.

In order to reduce stress and upsetness to a patient (particularly a child) when measuring vital signs, the present invention includes a flexible member that is coupled to one or more devices that measure vital signs. The one or more devices that measure vital signs provide data (corresponding to the measurements) to a processor located in the flexible member. The flexible member includes a wireless transmitter located therein for transmitting the data corresponding to the vital signs to a further microprocessor-based system outside of the flexible member.

In an exemplary embodiment of the present invention, the flexible member is a cloth member such as a blanket. The blanket has a desirable size so that it can be clutched and/or placed over the patient. In this manner, the cloth member is playing a psychological role, such as the role of a “security blanket.” In this manner, for example, a child may clutch the blanket and feel better about the fact that he/she needs to stay in a hospital over period of time. The blanket can also have a thermal effect which may allow the patient to stay warm. In this manner, the blanket is performing several objectives simultaneously, namely, providing psychological comfort to a patient while at the same time creating a location where various vital signs are aggregated and then data corresponding to those vital signs are transmitted to a microprocessor outside of the blanket.

In a further exemplary embodiment of the present invention, the flexible member may be a pillow, or may be included in a pillow, such as pillow filled with a compressible material such as foam or a plurality of feathers. In this manner, the flexible member can be grabbed, compressed, or used for padding, such as under a person's head or under a person's body while they are lying down.

In a further exemplary embodiment of the present invention, the flexible member has a surface to which objects may be adhered thereto. Exemplary objects may include toys, such as toys filled with compressible materials (“stuffed animals”), electronic toys, electronics, etc.

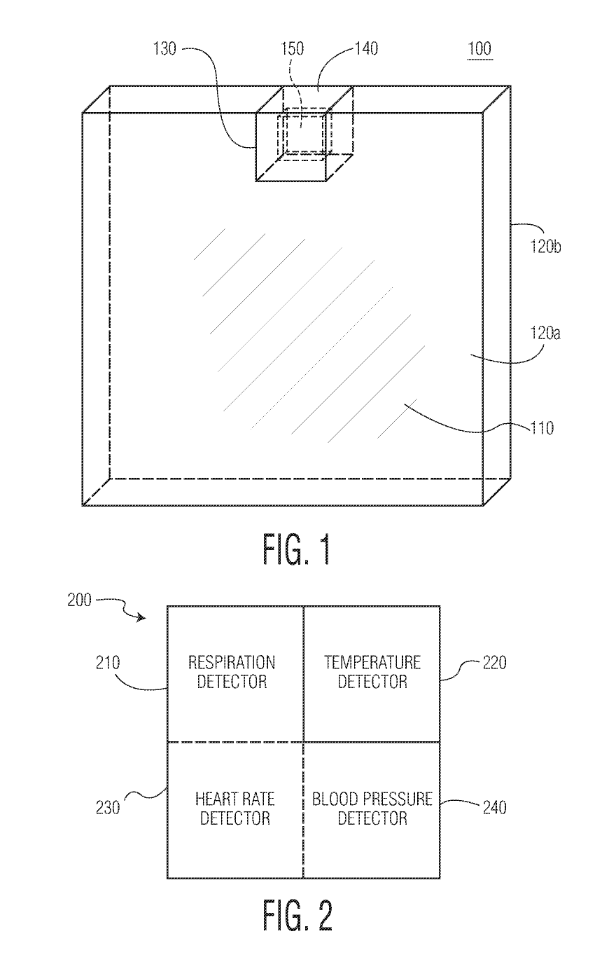

FIG. 1 illustrates an exemplary measurement apparatus 100 in accordance with an exemplary embodiment of the present invention. Flexible member 110 is shown and may be comprised of a plurality of materials such as a fiber-based material. A fiber-based material may use a natural substance such as cotton, an artificial substance such as polyester, or a combination thereof. Alternatively, the flexible member can be comprised of a nonwoven material such as plastic. Flexible member 110 includes pocket 130. Pocket 130 may hold a plurality of different objects as will be more clearly described below.

The size of flexible member 110 is larger than the size of pocket 130 that contains wireless transmitting device 150. In an exemplary embodiment of the present invention, flexible member 110 has a length and width that results in an area that is greater than area occupied by length and width of pocket 130. For example, FIG. 1 illustrates that pocket 130 occupies a portion of the area occupied by flexible member 110.

Pocket 130 is formed with pocket closure mechanism 140. Pocket closure mechanism 140 allows pocket 130 to be closed so that the contents therein are not easily removed or fall out. FIG. 1 illustrates that pocket 130 holes wireless transmitting device 150. As will be described below, wireless transmitting device 150 obtained data corresponding to various vital signs and transmits the data (or a process form thereof) to a microprocessor device outside of flexible member 110.

As shown in FIG. 1, Flexible member 110 may be rectangular, although this is merely exemplary. Flexible member 110 may be of many different sizes, although a minimum of 30 cm×30 cm is desirable. Flexible member 110 includes flexible member front 120A and flexible member back 120B. In one exemplary embodiment of the present invention, flexible member 110 has an internal space that may be filled with various objects. In one exemplary embodiment, flexible member 110 is filled with the compressible material so that flexible member 110 becomes a pillow or cushion. In another exemplary embodiment, flexible member 110 includes a heat insulating material therein in order to form a comforter. The heat insulating material can be formed of any material that is known to one of ordinary skill the art including cotton, wool, polyester, etc.

As shown in FIG. 2, measurement apparatus 100 also includes detectors 200 for measuring various types of vital signs. Exemplary detectors include respiration detector 210, temperature detector 220, heart rate detector 230, and blood pressure detector 240. As shown in FIG. 2, in some exemplary embodiments, it is possible that one detector may be used for measuring more than one vital sign. The details regarding each detector will be further explained below.

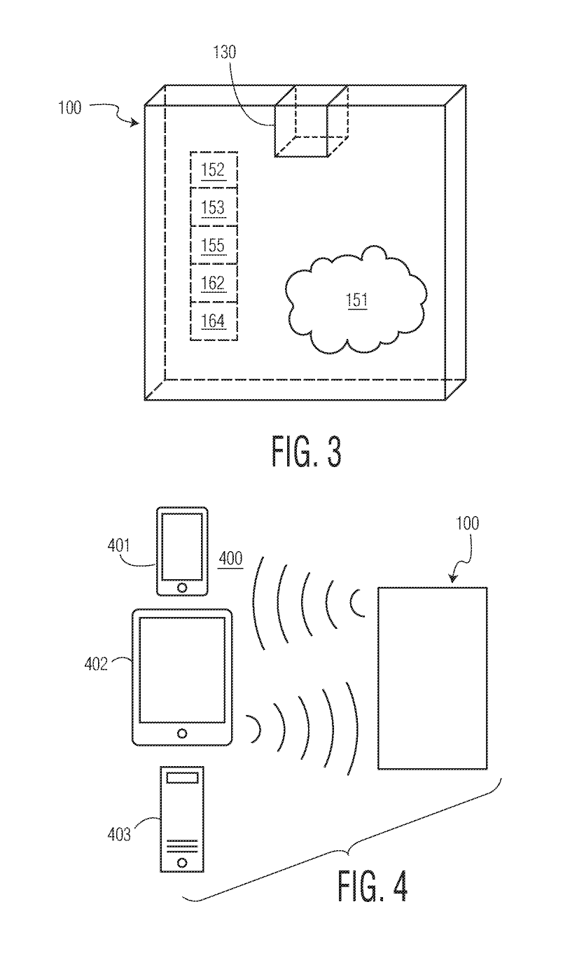

FIG. 3 illustrates exemplary embodiments of the present invention. In particular, various optional features of measurement apparatus 100 may be included. Exemplary optional features include speaker 152, vibrator 153, light 155, clock 162, and piezoelectric element 164. Speaker 152 may be included with measurement apparatus 100 for the purpose of allowing a patient to listen to various types of audio including music, soothing sounds, etc. Vibrator 153 may be included to provide a patient with a pleasant vibration motion. Vibrators are known, generally speaking, to one of ordinary skill the art, and may be implemented, for example, with a small motor, a shaft, and a weight attached to the end of the shaft so that the shaft rotates in an unbalanced manner. Light 154 may include one or more lights which may be used to provide pleasant aesthetics. Light 155 may be implemented, for example, using LED technology. Clock 162 may be a simple digital clock that can be used to allow a patient to see the current time. Piezoelectric element 164 may be used, for example, as an accelerometer in order to detect when measurement apparatus 100 is moved, thrown, dropped, etc.

FIG. 4 is a block diagram which illustrates how measurement apparatus 100 communicates with a microprocessor-based system outside of measurement apparatus 100. As shown in FIG. 4, various microprocessor-based devices are able to communicate with measurement apparatus 100. Exemplary devices include smart phone 401, tablet 402, and/or personal computer 403 which may be in various forms such as tower, laptop, etc. Measurement apparatus 100 is able to communicate wirelessly with a microprocessor-based computer system. In an exemplary embodiment of the present invention, communication is accomplished using a wireless communications format. Exemplary wireless communication formats include Wi-Fi, Bluetooth, NFC, cellular data, etc. A wired form of communication such as via USB or with ethernet cable may also be included (for example, as a backup).

### Body Temperature Detection

A conventional method of measuring core body temperature is accomplished via sublingual temperature measurement. Using this method, an oral thermometer is placed under a patient's tongue.

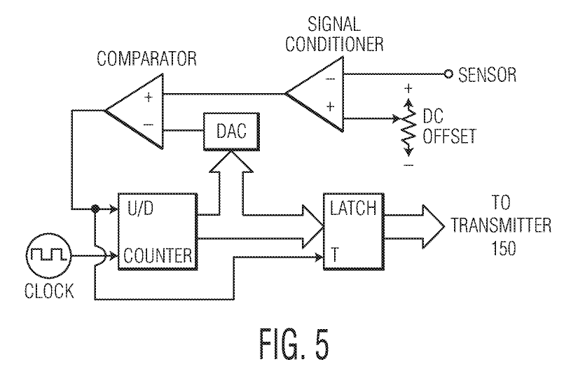

FIG. 5 illustrates an exemplary electronic thermometer. In this exemplary embodiment, the sensing element is a thermistor with a resistance to temperature range of approximately 180 K ohms at freezing to 2.5 K ohms at boiling. When connected in series with a 180 K ohm fixed resistor, the output voltage is a function of the thermistor resistance (or the temperature) is (V+) 180K/Rth+180K. This is the voltage divider equation. Vout is connected to the input of an LM324 operational amplifier set up with the voltage gain of 2 and a DC offset of −2.5 V. This tailors the analog voltage to go from 0 V to 5 V. The 0 to 5 volt analog signal goes into a voltage comparator where it is compared to a voltage generated by the binary count. An 8 up/down counter is outputted to an 8 bit data latch with the correct count will be latched into an optional display and simultaneously be transmitted to a digital to analog converter. This DAC is made out of resistors whose weight values are 1, 2, 4, 8, 16, 32, 64 and 128 times each other. They are followed by an operational amplifier in a voltage follower designed to prevent loading the network and distorting the output. This output goes to the other side of the voltage comparator. When the count generates a voltage that is slightly higher than the conditioned analog voltage, a flag out of the comparator latches the value into the display latching causes the counter now countdown until the county equivalent voltage drops below the conditions analog voltage. In this way, the ADC locks onto the analog signal and tracks it as it goes up or down.

The thermistor may be a 2K to 100K negative temperature coefficients thermal resistor.

The design set forth above is merely exemplary and it is understood that other methods of electronic temperature measurement may also be used.

In one exemplary embodiment of the present invention, temperature measurements from such electronic thermometer are taken by sensing data under a patient's tongue. The measured values are digitized and are transmitted to wireless transmitting device 150. Such transmission may be accomplished in accordance with known wireless communication methods that are known to one of ordinary skill in the art. Transmission of the temperature data may be accomplished over a wired path, although a wireless path is preferred. The transfer of temperature data from an electronic thermometer to wireless transmitting device 150 is accomplished using communication methods that are known to one of ordinary skill me art.

While sublingual temperature measurement is easy to accomplish and cost-effective, sublingual temperature measurement does not provide continuous ongoing temperature monitoring. Thus, there are other methods that may be used for continuous (or further) measurement of body temperature without having to hold a thermometer under patient's tongue. United States patent application US 2007/0295713 (Carlton-Foss) discloses one such exemplary method and is hereby incorporated by reference in its entirety. Carlton-Foss discloses a system and method for measuring core body temperature that includes a layered thermometer, including a plurality of layers. A 1^{st} layer contacts the skin of the body being measured. The 1^{st} layer includes a 1^{st} sensor and a 1^{st} insulating component, and optionally a protective layer contiguous with the patient's skin. The 1^{st} layer detects a 1^{st} temperature substantially at the skin. A 2^{nd} layer is located contiguous to the 1^{st} layer with a 2^{nd} layer includes a 2^{nd} sensor and a 2^{nd} insulating component. The 2^{nd} layer detects a 2^{nd} temperature substantially away from the skin. The values of the 1^{st} temperature and the 2^{nd} temperature indicate the core temperature of the patient's body.

In particular, Carlton-Foss discloses a thermometer comprised of a plurality of sensors, each arranged in a respective layer to create a thermometer array. The temperature sensors may be, for example, YS I 427 and 409 AC thermistor probes. The array of layers and thermistor probes creates a sandwich or telescope configuration although this is merely exemplary. Such a thermometer can be held close to a patient's skin by adhesive, belt, or strap. In this manner, temperature can be obtained on a continuous basis.

The above descriptions are merely exemplary of two alternative methods for measuring body temperature of a patient. Other methods for measuring body temperature of a patient are also known to one of ordinary skill me art. By obtaining electronic data that corresponds to body temperature, and transmitting that data to wireless transmitting device 150, wireless transmitting device 150, located within flexible member 110, is able to obtain a plurality of different types of vital signs for a patient.

Such body temperature detection may be accomplished via temperature detector 220 within an optional housing.

### Blood Pressure Detection

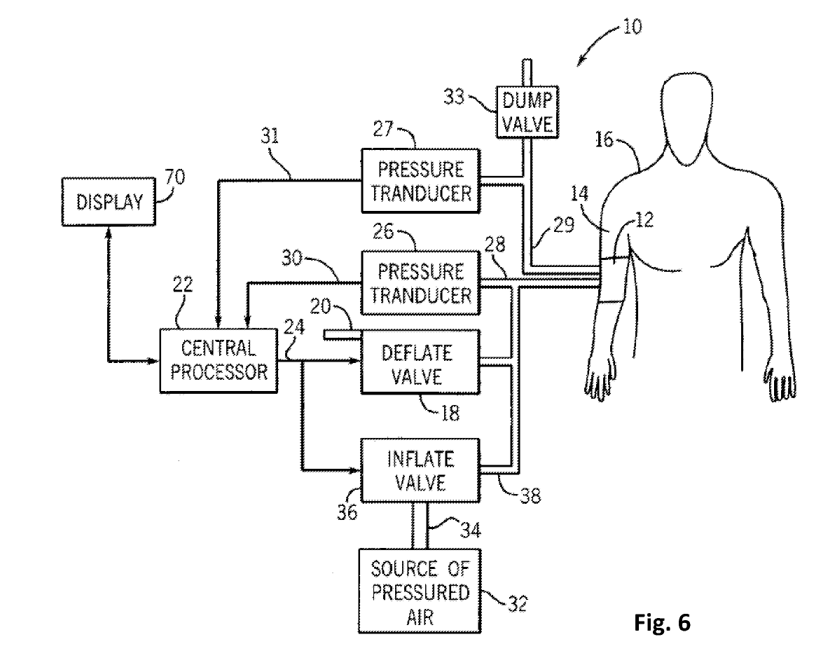

Blood pressure may be measured electronically in order to obtain digital data that corresponds to the current blood pressure of a patient (systolic and diastolic). In one exemplary embodiment of the present invention, blood pressure is determined using a method and apparatus disclosed in U.S. Pat. No. 7,153,269 (Blansett) which is hereby incorporated by reference in its entirety. FIG. 6 is a block diagram which illustrates the basic operation of an automated blood pressure detection system. The block diagram illustrates a noninvasive blood pressure (NIBP) monitoring system 10 of conventional construction. The NIBP monitoring system 10 includes a blood pressure cuff 12 placed on the arm 14 of a patient's 16. The blood pressure cuff 12 can be inflated and deflated for occluding the brachial artery of the patient 16 when in the fully inflated condition. As the blood pressure cuff 12 is deflated using the deflate valve 18 having exhaust 20, the arterial occlusion is gradually relieved. The deflation of the blood pressure cuff 12 by the deflate valve 18 is controlled by a central processor 22 through the control line 24.

A first pressure transducer 26 is coupled by duct 28 to the blood pressure cuff 12 for sensing the pressure within the cuff 12. In accordance with conventional oscillometric techniques, the transducer 26 is used to sense pressure oscillations in the cuff 12 that are generated by pressure changes in the brachial artery under the cuff. The electrical oscillation signals from the pressure transducer 26 are obtained by the central processor 22, using an analog-to digital converter, through connection line 30.

In the illustrated design, a second pressure transducer 27 is coupled by duct 29 to the blood pressure cuff 12 for sensing the pressure within the cuff 12. The duct 29 includes a dump valve 33 that can be opened to rapidly release the air pressure in the blood pressure cuff 12 to atmosphere. The electrical signals from the second pressure transducer 27 are also obtained by the central processor 22 through connection line 31. In the embodiment of the invention shown in FIG. 1, the first pressure transducer 26 and the second pressure transducer 27 are located in different areas of the NIBP monitoring system 10. The different positions of the pressure transducers 26, 27 within the NIBP monitoring system will result in different noise levels included in the oscillation signals output from the pressure transducers. For example, if one of the pressure transducers 26, 27 is located near the air compressor providing the pressurized air to the cuff, different noise levels will be present in the oscillation signal sent to the central processor 22.

A source of pressurized air 32, such as an air compressor or compressed gas cylinder, is connected by duct 34. In an embodiment incorporating an air compressor, the air compressor coupled directly to the duct 38. However, if the source of pressurized air is supplied by a compressed gas cylinder, an inflate valve 36 is positioned between the source 32 and the duct 38. The operation of the inflate valve 36 is controlled by the central processor 22 through the control line 24. Thus, the inflation and deflation of the blood pressure cuff 12 is controlled by the central processor 22 through the deflate valve 18 and the inflate valve 36, respectively.

The processing of the oscillation signals from first pressure transducer 26 and/or the second pressure transducer 27 by the central processor 22 to produce blood pressure data, and optionally to reject artifact data, can be conducted in accordance with the prior art teachings of the above-referenced Ramsey '029 and '034 patents. Alternatively, the blood pressure can be determined in accordance with the teachings of Medero et al in U.S. Pat. No. 4,543,962, of Medero in U.S. Pat. No. 4,546,775, of Hood, Jr. et al in U.S. Pat. No. 4,461,266, of Ramsey, III et al in U.S. Pat. No. 4,638,810, of Ramsey III et al in U.S. Pat. No. 4,754,761, of Ramsey III et al in U.S. Pat. No. 5,170,795, of Ramsey III et al in U.S. Pat. No. 5,052,397, of Medero in U.S. Pat. No. 5,577,508 and of Hersh et al in U.S. Pat. No. 5,590,662, all of which are hereby incorporated by reference in their entirety. In any event, it is desirable to use any of the known techniques to determine the quality of the oscillation complexes received at each cuff pressure so that the blood pressure determination is made using the physiological relevant cuff pressure oscillations from each heart beat and not artifacts.

While the use of a cuff to accomplish the measurement of blood pressure is easy to accomplish and cost-effective, the use of a cuff for ongoing blood pressure measurement may be less desirable than other methods of blood pressure detection, particularly when continuous ongoing blood pressure monitoring is desired. Thus, there are other methods that may be used for continuous (or further) measurement of blood pressure without having to maintain a patient's arm in a cuff. U.S. Pat. No. 5,857,975 (Golub) discloses one such exemplary method and is hereby Incorporated by reference in its entirety. Golub discloses a method and apparatus for non-invasive, cuffless continuous blood pressure determination. Arterial blood pressure of a subject is determined by detecting the EKG for the subject and selecting a fiducial point on the EKG during a pulse. Apparatus is provided for monitoring blood volume versus time at a selected location on the subject's body such as a fingertip. A time difference between the occurrence of the selected fiducial point on the EKG and a selected change in blood in volume at the selected body location is determined. This time difference depends on the arrival time of the pulse at the distal location in addition to the shape of the blood volume versus time curve. Heart rate is determined from the EKG. The arterial pressure is computed from pulse arrival time, volumetric wave shape and instantaneous heart rate for each pulse. It is preferred that the fiducial point on the EKG be an R-wave. A suitable method for determining change in blood volume utilizes photoplethysmography. Methods are disclosed for determining diastolic pressure, systolic and mean arterial pressure. In another aspect, artifact detection and rejection enabled.

U.S. Pat. No. 4,331,154 (Broadwater) discloses another such exemplary method and is hereby incorporated by reference in its entirety. A digital watch is employed to measure systolic and diastolic blood pressure as well as heart rate. The band of the watch supports a piezoelectric transducer that is held in contact with the wrist adjacent to the radial artery when a switch on the band is activated. The watch contains electronic circuitry that derives a binary representation of the maximum or systolic pressure and the minimum or diastolic pressure that is generated at the artery by blood pressure pulses which can be displayed. Electronic circuitry is also provided to generate and address corresponding to the time interval T between successive measured heart beats. The address is used to access a memory device that contains a stored list of heart rates in cycles per minute. The contents of the memory at the address T corresponds to the reciprocal of T, in units of pulses per minute and a representation of the contents of the memory can be displayed.

U.S. Pat. No. 6,413,223 (Yang) discloses another such exemplary method and is hereby incorporated by reference in its entirety. Noninvasive monitoring devices such as finger photoplethysmographs and an electrical impedance photoplethysmograph (EIP) to monitor the dynamic behavior of the arterial blood flow. In this approach, measured signals from these noninvasive sensors on an arterial segment are integrated to estimate the blood pressure in the segment based on a hemodynamic model. A mathematical model of the arterial blood flow is derived and transformed into a state-space representation. In the modeling, a precise hemodynamic model for the arterial segment on which sensors are located is derived, and combined with relatively simplified models of the upstream and the downstream arterial flows to represent an entire arterial stream. Then, a Kalman filter is designed based on the model and it is shown that the internal variables such as the arterial blood pressure in the arterial segment can be estimated based on the measurements, even though the observability condition of the system may not be met. Simulation results indicate that the approach can generate an accurate estimation of the arterial blood pressure in real-time even from noisy sensor signals.

The above descriptions are merely exemplary of alternative methods for measuring body temperature blood pressure of a patient. Other methods for measuring blood pressure of a patient are also known to one of ordinary skill me art. By obtaining electronic data that corresponds to blood pressure, and transmitting that data to wireless transmitting device 150, wireless transmitting device 150, located within flexible member 110, is able to obtain a plurality of different types of vital signs for a patient.

Such blood pressure detection may be accomplished via blood pressure detector 240 within an optional housing.

### Heart Rate Monitor

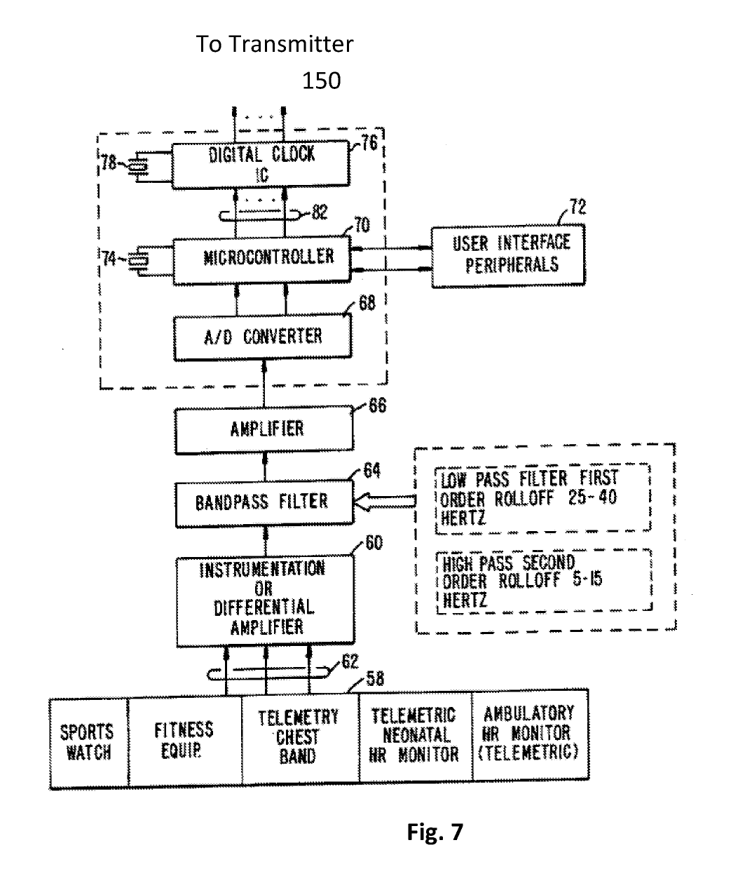

FIG. 7 illustrates an exemplary heart rate monitor as disclosed by U.S. Pat. No. 5,738,104 which is hereby incorporated by reference in its entirety. Block 58 represents the electrical contacts. Block 60 is a differential amplifier or instrumentation amplifier that is coupled via bus 62 to the contacts. The differential amplifier amplifies the analog signals on the three conductors of bus 62. The common input on bus 62 is coupled to analog ground and the two remaining conductors are coupled to the plus and minus inputs of the differential amplifier. The differential amplifier serves to provide gain and to simultaneously eliminate common mode noise in the signal such 60/50 Hertz hum etc. The gain of the differential amplifier is set at a relatively low figure of 5-10 to prevent saturation of the operational amplifiers therein by the low frequencies that are still in the analog signal.

Next, the output of the amplifier is filtered by an active analog bandpass filter 64. Active filters using RC components in operational amplifier circuits are used to implement the bandpass filter. The passband of this filter is centered on some frequency between 5 and 20 Hertz, preferably 10-15 Hertz, and has a passband of approximately 5-40 Hertz. The purpose of this bandpass filter is to remove high frequencies from the analog signal to prevent aliasing. The low frequency corner of the passband also eliminates DC components and any low frequency drift of the baseline caused by respiration muscle contractions or other muscle contractions such as typically occur when the user is exercising. The gain of the bandpass filter is preferably set at 1.5 at the center frequency, but that gain figure is not critical. A passive bandpass filter could be used, but generally is not preferred since it requires too many components and the rolloff is not sharp enough. In the preferred embodiment, the passband filter is comprised of two separate hardware filters: a low pass filter with a first order rolloff and a corner frequency between 25-40 Hertz; and a high pass filter with a second order rolloff with a corner frequency of from 5-15 Hertz. The order in which these two filters filter the incoming signal from the differential amplifier is not important. The reason these two filters have different order rolloffs is to allow the differentiator to emphasize the high frequency components in the signal in a manner described below. In other words, the steeper rolloff on the low frequency corner provides better selectivity, while the less steep rolloff on the high frequency corner allows more high frequency component to reach the differentiator such that the differentiator will generate bigger slope numbers because of the sharp peaks in the signal represented by the data reaching the differentiator because of the higher content of high frequency components in the analog signals reaching the analog-to-digital converter. These sharper corners cause the differentiation operation in the digital signal processing portions of the circuit to be described below to generate higher peaks. These higher peaks are filtered out by the moving average function, but in the process, the average rises thereby causing the EKG signals to stand out better against the background noise.

Next, the analog signal is amplified in amplifier 66 which has a gain of 100-1000 so that the overall gain is about 1000-10,000.

Next, the analog output of the amplifier 66 is applied to the input of an analog-to-digital converter (hereafter AD converter) 68, which, in the preferred embodiment is integrated onto the microcontroller integrated circuit. The analog signal is converted to 8-bit digital samples at a sampling rate of 180 Hertz, or some other multiple of 60 Hertz preferably. Other sampling rates can also be used so long as the high frequency components of the real signal from the electrodes have been filtered out prior to the real signal reaching the analog-to-digital converter. If these high frequency components have not been filtered out, alias signals will appear in the EKG frequency range resulting from beating of the sampling rate with the high frequency components. This is why use of an analog bandpass filter in front of the analog-to-digital converter is preferred. Different numbers of bits in each sample could be used as well as a higher or lower sampling rate, but 180 Hertz is preferred to make the digital filtering easier since the 180 Hertz sampling rate is a multiple of the 60 Hertz frequency of a common source of noise. Further, the coefficients for the digital signal processing stages described below are set for a sample rate of 180 samples per second, so if other sample rates are used, the coefficients must be optimized again. In some embodiments, the AD converter is part of a microcontroller integrated circuit 70. The AD converter should be able to operate on a 3 volt supply or some supply voltage easily achievable with batteries and, for size considerations in some embodiments, preferably has a serial format output data stream if the AD converter is not integrated onboard the microcontroller.

The digital data stream from the AD converter is input to the microcontroller 70 for further signal processing to be described below. The microcontroller is preferably a 4-bit or 8-bit machine such as a Samsung KS57C2408 or Hitachi H8/3812 or equivalent. These microcontrollers have on-board analog-to-digital converter, dual clocks, LCD drive and buzzer drives. There is no need for an on-board DSP or a separate DSP since the digital filter coefficients are integers which substantially simplifies and speeds up processing in the software filtering modules.

The above description is merely exemplary and other methods and apparatus are known for measuring heart rate. Examples include U.S. Pat. No. 6,413,223, U.S. Pat. No. 8,768,424 and U. S. Application 2014/0107493, all of which are incorporated by reference in their entireties.

The above descriptions are merely exemplary of alternative methods for measuring heart rate of a patient. Other methods for measuring heart rate of a patient are also known to one of ordinary skill in the art. By obtaining electronic data that corresponds to heart rate, and transmitting that data to wireless transmitting device 150, wireless transmitting device 150, located within flexible member 110, is able to obtain a plurality of different types of vital signs for a patient.

Such heart rate detection may be accomplished via heart rate monitor 230 within an optional housing.

### Respiration

Methods of measuring respiration are varied and are known to one of ordinary skill in the art. One example is disclosed in U.S. Pat. No. 4,308,872 which is hereby incorporated by reference in its entirety. Other methods are also known including methods and apparatus that are disclosed in U.S. Application 2015/0250392 and U.S. application Ser. No. 13/452,632 which are hereby incorporated by reference in their entireties. Pulse Oximetry is also known to one of ordinary skill in the art as a method of measuring respiration and may be used in combination with the present invention. Pulse oximetry is normally accomplished using a pair of LEDs facing a photodiode through a translucent part of the patient's body, such as a finger or earlobe. The LEDs have wavelengths of 660 nm and 940. The percentage of blood oxygen is calculated based on the absorption rate from each wavelength after it passes through the patient's body. See “Pulse Oximeter uses ADuC7024 MicroConverter” at www.analog.com which is hereby incorporated by reference in its entirety.

Such respiration detection may be accomplished via respiration detector 210 within an optional housing.

Therefore, one or more of the plurality of detectors 200 collect vital sign data and transmit the vital sign data so that the vital sign data can be seen, recorded, evaluated, etc. As described with reference to FIG. 4, the obtained vital sign data may be transmitted to a microprocessor based device such as smartphone 401, tablet 402, or personal computer 403 (for example). Alternatively, the vital sign data may be transmitted directly to a network for later receipt by a microprocessor based device.

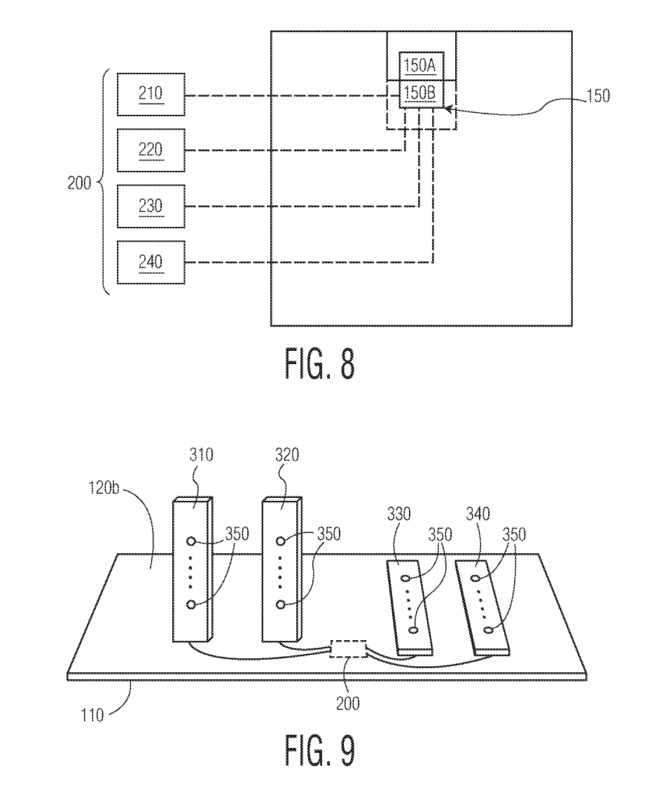

Wireless transmitting device 150 is comprised of several components as shown in FIG. 8. One component of wireless transmitting device 150 is power source 150a. Power source 150a may be a battery, such as a rechargeable NiCd battery. Transmitting device 150 also includes transmitting unit 150b. Transmitting unit 150b receives vital sign data provided by any of the detectors 200 and transmits the data of a microprocessor device external to flexible member 200 (for example, devices, 401, 402, and 403). Transmitting unit 150b is comprised of at least two components including a data transmitter and a microcontroller to control the data transmitter. In a preferred embodiment of the present invention, the data transmitter transmits using Bluetooth data format, although this is merely exemplary, and other forms of data transmission may be used including for example, cellular, wi-fi, NFC, etc. An exemplary data transmitter may be the HC-06 serial Bluetooth transceiver, manufactured by Guangzhou HC information Technology Co. Ltd, Guangzhou, China, and sold through distributors such as Amazon. Operation of the HC-06 is accomplished based on the HC-06 datasheet obtained at www.wavesen.com, under HC-06 module datasheet (English) which is hereby incorporated by reference in its entirety. The HC-06 may be operated by a microcontroller such as the Arduino Lino R3 Microcontroller A000066, manufactured by Arduino, and available through sources such as Amazon. Operation of the A000066 controller is accomplished via datasheet incorporated by reference herein in its entirety and available at www.arduino.cc. Integration of the two may be accomplished by following Arduino Bluetooth Basic Tutorial available at www.instructables.com and incorporated by reference herein in its entirety.

FIG. 8 also illustrates that in some exemplary embodiments, only portion of wireless transmitting device 150 is located in pocket 130. In the embodiment shown in FIG. 1, substantial portions (i.e. more than half or all) of wireless transmitting device 150 is located in pocket 150. In the embodiment shown in FIG. 8, power supply 150a is located in pocket 130 and transmitting unit 150b is located outside of pocket 130 (but within flexible member 110). In the exemplary embodiment shown in FIG. 8, the battery may easily be removed for recharge/replacement. In the embodiment shown in FIG. 1, wireless transmitting device 150 is removed for recharge (and/or at least partial replacement).

In the embodiment shown in FIG. 8, each of the detectors are separated from flexible member 110, and are connected to flexible member 110 for communication, power, or both. In one embodiment, there are cables situated between each of the detectors 200. The cables extend from each detector 200 to transmitting device 150 situated in flexible member 110. The cables may be communication cables, power cables, or both. The connections shown in FIG. 8 are shown in dashed lines to indicate that these connections may be via cables, or, in some exemplary embodiments may be wireless. In such embodiments, each (or some) detector 200 has its own power source (or shares a power source with another detector 200). Alternatively, or in addition, in some embodiments, each (or some) detector 200 communicates with transmitting device 150, and sends respective vital sign data to transmitting device 150 via a wireless communication protocol including (but not limited to) cellular, Bluetooth, Wi-Fi, and NFC.

FIG. 9 illustrates a further exemplary embodiment of the present invention, in which detectors 200 are integrated into flexible member 110. As shown in FIG. 9, one or more extensions protrude from flexible member 110. Belt 310, for example, may be cuff 12 that is used in combination with blood pressure detector 240 for measuring blood pressure. Belt 320 may include sensors that are used in combination with heart rate detector 230 for measuring heart rate. Belt 330 may include sensors (including LEDs and a photodetector used in pulse oximetry, for example) that are used in combination with respiration detector 210 for measuring respiration rate. Belt 340 may include sensors that are used in combination with temperature detector 220 for measuring body temperature. While four belts are shown, this is merely exemplary, as the actual number included may vary from less than four (including zero) to more than four. One belt may perform multiple types of detections. Also, each belt 310, 320, 330, 340 are shown in various configurations relative to bottom surface 120b of flexible member 110, but this is merely exemplary. For example, belts 310, 320 are shown in a configuration where portions thereof separate from bottom surface 120b, while belts 330, 340 are shown flush with bottom surface 120b. Again, this is completely exemplary as at least some (or no) belts may be partially separatable from bottom surface 120b, and at least some (or no) belts may be flush with bottom surface 120b. In such an exemplary embodiment, wrapping flexible member 110 around the patient causes any of the belts 310, 320, 330 and/or 340 to be wrapped around the patient. In this manner, sensors 350 (and associated electronics) can transmit signals to respective detectors 200 so that vital sign measurements can be obtained and those measurements can be converted into data that is transmitted to transmitting device 150.

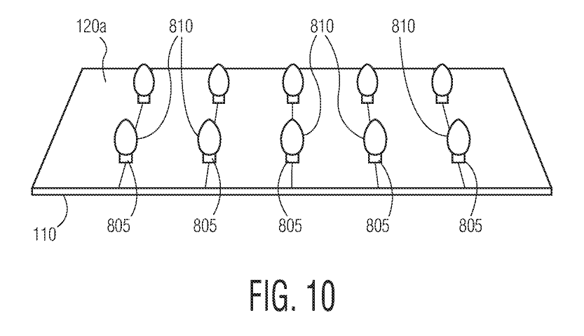

FIG. 10 illustrates a further exemplary embodiment of the present invention in which objects, such as one or more objects 810 are adhered to an outer surface of flexible member 110. Objects 810 may be various types of objects including, for example, toys such as plush stuffed animal toys. Attachment (e.g. “hook and eye”) surfaces 805 may be attached to flexible member front 120a. Objects 810 may have corresponding (e.g. “hook and eye”) surfaces that adhere to “hook and eye” surfaces 805. By using “hook and eye” surfaces, objects 810 may be easily attached to and subsequently removed from flexible member front 120a. The use of “hook and eye” surfaces is merely exemplary, as other forms of attachment may be used to secure objects 810 to flexible member front surface 120a, such as clips, buttons, snaps, pins, magnets, friction, etc.

Flexible member 110 is shown by itself in several figures, but this is merely exemplary. Flexible member 110 may be inserted into another (e.g. cloth) member, such as a member with an interior, so that flexible member 110 is used as part of a soft object such as a pillow. Flexible member 110 may be inserted into a pillow case that also includes compressible materials, such as a plurality of feathers, or foam. In such a configuration, flexible member 110 may be included in a pillow (for example) and may be positioned anywhere that comfort is desired, such as under a person's head or under another body part(s).

The above descriptions are merely exemplary of alternative methods for measuring respiration of a patient. Other methods for measuring respiration of a patient are also known to one of ordinary skill in the art. By obtaining electronic data that corresponds to respiration, and transmitting that data to wireless transmitting device 150, wireless transmitting device 150, located within flexible member 110, is able to obtain a plurality of different types of vital signs for a patient.

While the present invention has been described herein with reference to exemplary embodiments, it should be understood that the invention is not limited thereto. Those skilled in the art with an access to the teachings herein will recognize additional modifications, applications, and embodiments within the scope thereof and additional fields in which the invention would be useful.

Embodiments of the invention also may be directed to computer program products comprising software stored on any computer useable medium. Such software, when executed in one or more data processing device, causes a data processing device(s) to operate as described herein. Embodiments of the invention employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nano-technological storage device, etc.).

The present application has set forth one or more but not all exemplary embodiments of the present invention as contemplated by the inventor(s), and thus, is not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein, it is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A vital sign measurement apparatus, comprising:
a flexible member that includes an interior;
said flexible member including a pocket;
said pocket including a closure mechanism for closing said pocket and maintaining said pocket in a closed position, said closure mechanism openable for accessing an interior of said pocket;
a wireless transmitting device at least partially located in said pocket and removable from said pocket upon opening said closure mechanism;
one or more detectors for measuring vital signs that includes at least one of respiration, body temperature, heart rate and blood pressure, wherein said one or more detectors communicate with said wireless transmitting device;
said pocket closed off from an interior of said flexible member outside of said pocket;
said pocket smaller than outer dimensions of said flexible member;
said wireless transmitting device includes a power source and a data transmitter that includes a microcontroller to control the data transmitter, wherein said data transmitter transmits data received from said one or more detectors located outside of the pocket to a computing device outside of said flexible member, wherein said power source is accessible for removal from said pocket after opening said closure mechanism;
opening said closure mechanism provides access to an interior of said pocket without also providing access to said interior of said flexible member outside of said pocket;
wherein said wireless transmitting device provides a wireless signal to a microprocessor based computer system external to said flexible member, said wireless signal corresponding to said vital signs that includes more than one of respiration, body temperature, heart rate, and blood pressure measured by said detectors and provided to said wireless transmitting device;
at least one of said detectors is at least partially on an outside of said flexible member for communicating vital signs to said wireless transmitting device that is at least partially in said pocket.

2. A vital sign measurement apparatus according to claim 1, wherein said flexible member includes a compressible material included in said interior to form a pillow.

3. A vital sign measurement apparatus according to claim 1, wherein said flexible member forms a blanket.

4. A vital sign measurement apparatus according to claim 1, wherein said flexible member includes a heat insulating material included in said interior to form a comforter.

5. A vital sign measurement apparatus according to claim 1, wherein said wireless transmitting device includes a receiver for receiving a further signal.

6. A vital sign measurement apparatus according claim 5, wherein said apparatus includes an audio speaker for generating audio that is based on said further signal.

7. A vital sign measurement apparatus according to claim 5, wherein said apparatus includes a vibrator that is actuated responsive to said further signal.

8. A vital sign measurement apparatus according to claim 1, wherein said wireless transmitting device is a Bluetooth device.

9. A vital sign measurement apparatus according to claim 1, wherein a light is attached to an outside of said cloth member.

10. A vital sign measurement apparatus according to claim 1, wherein sensors are attached to said loop member and face interior to said loop member.

11. A vital sign measurement apparatus according to claim 1, wherein a clock is attached to said cloth member and said clock displays time externally to said cloth member.

12. A vital sign measurement apparatus according to claim 1, wherein said wireless transmitting device includes a piezo electric element for detecting vibration.

13. A vital sign measurement apparatus according to claim 1, further comprising an attachment portion on an exterior of said cloth member for attaching an external object to said attachment portion.

14. A vital sign measurement apparatus according to claim 1, wherein said microprocessor based computer system is a smartphone or tablet that displays on its screen data corresponding to said breathing, temperature, heart rate, and blood pressure.

15. A vital sign measurement apparatus according to claim 1, further comprising a proximity detector for detecting location of said vital sign measurement apparatus.

16. A vital sign measurement apparatus according to claim 1, wherein said flexible member is comprised of cloth.

17. (canceled)

18. A vital measurement apparatus according to claim 1, wherein said at least one detector measures at least one of heart rate and blood pressure.

19. A vital sign measurement apparatus according to claim 1, wherein one of said detectors measures heart rate and includes an amplifier for amplifying vital signs obtained by said one of said detectors and a filter for reducing noise in a signal generated in said amplifier that originated from said one of said detectors.

20. A vital sign measurement apparatus according to claim 1, wherein said flexible member is a) a pillow-like member or b) a blanket that includes an insulating layer.

21. A vital sign measurement apparatus according to claim 1, wherein area of said flexible member that extends to boundaries of said pocket is smaller than area of said flexible member beyond where said boundaries of said pocket extend.
